(19) United States
(12) Patent Application Publication
(10) Pub. No.: US 2016/0143762 A1
(43) Pub. Date: May 26, 2016

(54) Ergonomic Hand Pillow

(71) Applicant: Satwant Shottha, Vancouver (CA)

(72) Inventor: Satwant Shottha, Vancouver (CA)

(21) Appl. No.: 14/922,253

(22) Filed: Oct. 26, 2015

Related U.S. Application Data
(60) Provisional application No. 62/083,607, filed on Nov. 24, 2014.

(51) Int. Cl. A61F 5/01; A47B 21/03; F16M 13/04

(52) U.S. Cl. CPC A61F 5/0118; F16M 13/04; A47B 21/0371

(57) ABSTRACT

The invention is directed toward an ergonomic hand pillow worn by a user to support the user's hand during repetitive tasks. The wearable device comprises a hand support and an attachment means. The hand support comprises a compressible material and has a first end and a second end. The first end of the hand support is substantially positioned under the wrist of a user when the hand support is worn by a user. The second end of the hand support is substantially positioned under the heads of the metacarpal bones when the hand support is worn by the user. The attachment means removably secures the hand support to the palm of a user's hand. The hand support may be straight or may be tapered in thickness and width.

### TECHNICAL FIELD

The invention relates generally to a write support device and more specifically to a personal wrist support device attached to the hand of the user to support the user's wrist during repetitive tasks which are likely to cause carpal tunnel syndrome.

### BACKGROUND OF THE INVENTION

People use computers for hours on end. The use of the computers and computer accessories can cause stress on the human body. People spend lots of time typing on computer keyboards. Repetitive movement and from typing can cause stress and pain in a user's hand or wrist. Additionally, a user may have to use a mouse or other computer accessory which may cause continued stress. Over time, stress and pain may become carpal tunnel syndrome. Supportive devices have been developed to support user's hands during the typing process. Soft gel devices can be placed at the base of a keyboard or next to a computer mouse to provide support for a user and provide relief to those who suffer from carpal tunnel syndrome.

Other repetitive movements and activities may exacerbate stress and pain in the user's wrist. Activities such as ironing may cause additional pain and discomfort. Other daily chores and activities where a user is performing a repetitive task can cause carpal tunnel syndrome or enflame a user's symptoms. Supportive structures developed for keyboards are not intended for use in other locations. Also, people now own multiple computers and devices. Many devices are now portable and can be used anywhere. Standard supportive devices are limited in that they are normally intended for use on a stationary device so a user may not always have the supporting structure. Users may forgot to grab the supporting structure and move it to a new location. What is needed is a completely portable device used to provide support to a user's wrist regardless of what the user is doing.

The present invention provides a benefit over existing devices in that it is utilized to support the wrist of a user during any activity in which the user is engaged in. The device may be utilized when the user is engaged in office activities such as typing or filing. Alternatively, the device may be utilized for domestic or home activities such as cooking or ironing. A user can utilize the device to protect and support the wrist while using scissors to cut paper or fabric during sewing activities or when crocheting. Thus, the portability of the present invention permits the user to protect their wrist and ease their pain regardless of the activity in which the user is engaged in.

### SUMMARY OF THE INVENTION

The invention is directed toward an ergonomic hand pillow worn by a user to support the user's hand during repetitive tasks. The invention is directed toward a wearable device for supporting the wrist of a user during a repetitive task comprising a hand support and an attachment means. The hand support comprises a compressible material and has a first end and a second end. The first end of the hand support is thicker and wider than the second end of the hand support. The hand support is substantially tapered from the first end to the second end. The first end of the hand support is substantially positioned under the wrist of a user when the hand support is worn by a user. The second end of the hand support is substantially positioned under the heads of the metacarpal bones when the hand support is worn by the user. The attachment means removably secures the hand support to the palm of a user's hand.

In another embodiment the attachment means comprises one or more looped bands. Additionally, the compressible material may be selected from a group consisting essentially of cotton, sponge, synthetic foam, gel, natural fibers, or thermoplastic elastomer. In another embodiment, the first end of the hand support is approximately 0.75 inches thick and approximately 3.5 inches wide. The second end of the hand support is approximately 0.25 inches thick and approximately 2.5 inches wide. The length of the hand support from the first end to the second end is between approximately 3.0 inches and approximately 3.5 inches. In another embodiment of the invention, the invention may further comprise a securing means. The securing means secures portions of the attachment means together or secures the attachment means to the hand support. The securing means may be selected from a group consisting essentially of hook and loop tape, a snap, a button, and a hook and eye.

In another embodiment the attachment means consists of two elastic looped bands. The two elastic looped bands encircle the back of a user's hand when worn by a user. The two elastic looped bands overlap at one point on the back of a user's hand.

In an alternative embodiment, the invention is directed toward a wearable device for supporting the wrist of a user during a repetitive task comprising a hand support and an attachment means. In this embodiment the hand support comprises an outer cover and a compressible inner material. The hand support has a first end and a second end. The first end of the hand support is thicker and wider than the second end of the hand support. The hand support is substantially tapered from the first end to the second end. The first end of the hand support is substantially positioned under the wrist of a user when the hand support is worn by a user. The second end of the hand support is substantially positioned under the heads of the metacarpal bones when the hand support is worn by the user. The attachment means removably secures the hand support to the palm of a user's hand. The compressible inner material may be selected from a group consisting essentially of cotton, sponge, synthetic foam, gel, natural fibers, or thermoplastic elastomer. Additionally, the outer cover may be composed of a material selected from a group consisting essentially of a cotton fabric, synthetic fabric, or synthetic rubber. In another embodiment, the attachment means may comprises one or more looped bands. In another embodiment of the alternative version, the first end of the hand support is approximately 0.75 inches thick and approximately 3.5 inches wide. The second end of the hand support is approximately 0.25 inches thick and approximately 2.5 inches wide. The length of the hand support from the first end to the second end is between approximately 3.0 inches and approximately 3.5 inches. The alternative version may further comprise a securing means. The securing means secures portions of the attachment means together or secures the attachment means to the hand support. The securing means may be selected from a group consisting essentially of hook and loop tape, a snap, a button, and a hook and eye.

In another embodiment of the alternative version the attachment means consists of two elastic looped bands. The two elastic looped bands encircle the back of a user's hand when worn by a user. The two elastic looped bands overlap at one point on the back of a user's hand.

### BRIEF DESCRIPTION OF THE DRAWINGS

FIG. 1 is a perspective view of the hand pillow on the hand of a user.

FIG. 2 is a perspective view of a user's hand displaying a portion of the hand pillow.

FIG. 3 is a side view of the hand pillow.

FIG. 4 is a cut away view of the hand pillow.

FIG. 5 is a perspective view of an alternative embodiment of the hand pillow on the hand of a user.

FIG. 6 is a perspective view of a user's hand displaying a portion of the hand pillow.

### DETAILED DESCRIPTION OF THE DRAWINGS

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this invention and the claims. Although the invention will be described with regards to the preferred embodiment, it should be self-evident to one having skill in the art that other variations and possibilities exist without departing from the scope of the invention.

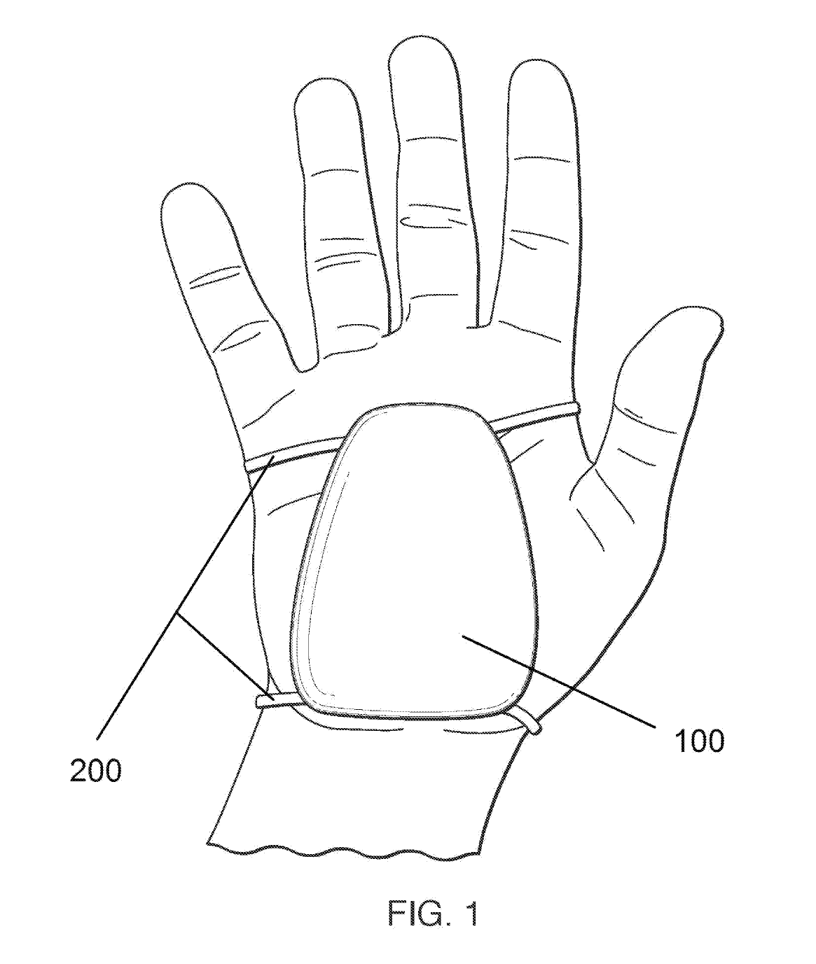

Referring to FIG. 1, the portable hand pillow is displayed. The hand pillow is comprised of a hand support 100 and attachment means 200. The hand support 100 is an ergonomically shaped support pillow that is smaller than the user's hand. The attachment means 200 attaches the hand support 100 to the user's hand. As displayed in FIG. 1 the hand support 100 is removably secured to the user's hand during use. The attachment means 200 secure the hand support 100 in the proper position to ensure that the hand support 100 provides the greatest benefit to the user. The hand support 100 is positioned so that the hand support 100 extends from the user's wrist to approximately three-quarters up the palm of the user's hand.

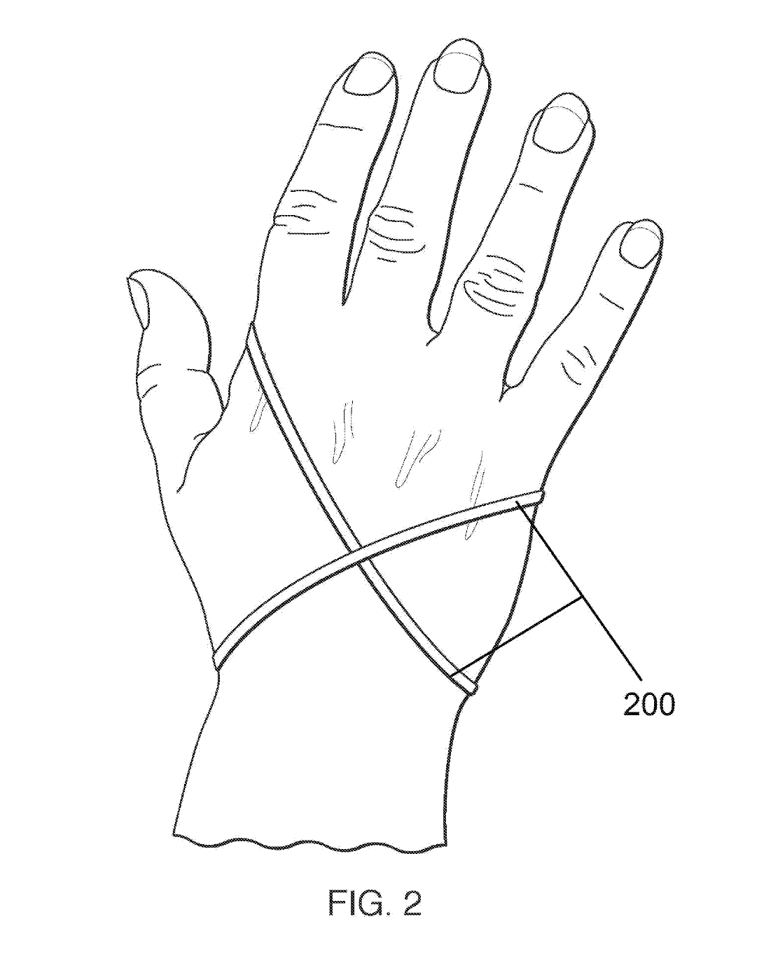

Referring to FIG. 2, the attachment means 200 are displayed on the back of the user's hand. The attachment means 200 can take any size or shape. In the preferred embodiment the attachment means 200 are a pair of looped bands that encircle the user's hand. In the preferred embodiment the looped bands cross each other to form an “X” on the back of the hand of the user. In other embodiments the attachment means 200 may comprise any number of looped bands. The looped bands may or may not cross each other. The looped bands may be composed of any type of material. The looped bands may be composed of strings, elastic bands, ribbon, fabric, or any other type of material. The looped bands may be complete circular bands or may be one or more band segments. One band segment may form a looped band by having each end of a band segment attached to the hand support 100. Alternatively two band segments may form looped bands by having one end of each band segment attached to the hand support 100 and the free end of each band segment temporarily secured to each other.

In some embodiments the device may further comprise a securing means (not shown). The securing means is utilized to secure two free ends of two band segments together, secure one or more free ends of a band segment to the hand support 100, or secure a circular loped band to the hand support 100. The securing means may be hook and loop tape, a snap, a button, a hook and eye, or any other device utilized to temporarily secure two items together. In some embodiments the securing means 200 may require a user to secure the attachment means 200 to the user's hand. For instance, a user may be required to tie two strings together, snap a button, secure two sections of hook and loop tape, secure a hook and eye together, or any other known means of securing items together, to secure the attachment means 200 together. The attachment means 200 may be positioned at any location on the user's hand. The attachment means 200 may wrap around the body of the user's hand. Alternatively, the attachment means 200 may wrap around one or more of the user's fingers. Alternatively, the attachment means 200 may wrap around the user's wrist. The attachment means 200 may also be positioned in any combination of these positions.

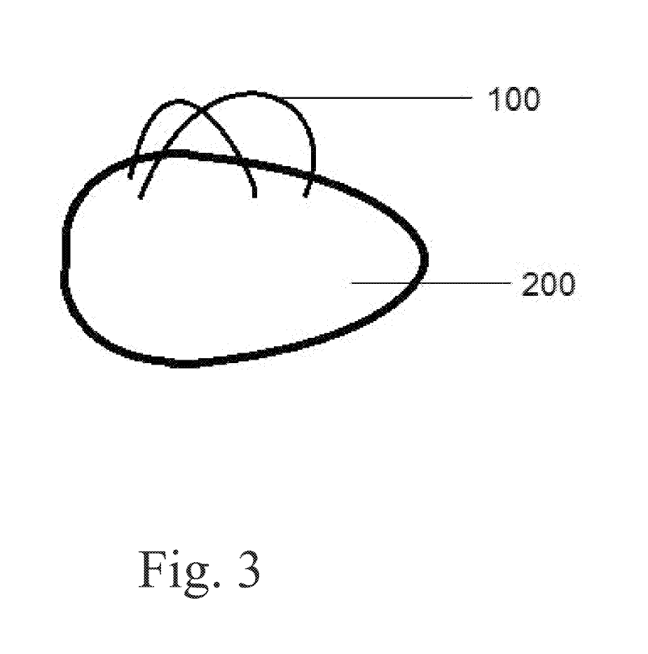

Referring to FIG. 1 and FIG. 3, the shape of the hand pillow is further illustrated. The hand support 100 is ergonomically shaped to have two distinct ends along its longitudinal axis. The first end of the hand support 100 is wider and thicker than the second end of the hand support 100. The hand support 100 is tapered in shape so that the first end provides maximum support to the user's wrist when worn. Additionally, the width of the hand support 100 is tapered so that the first end of the hand support 100 is wider than the second end of the hand support 100. The tapered shape of the hand support 100 allows for the user's wrist to be properly supported at the thicker and wider end while remaining out of the user's way and not interfering with the user's fingers at the thinner and narrower end. In the preferred embodiment, the hand support 100 extends from the user's wrist, under the user's palm, to the end of the user's metacarpal bones. The metacarpal bones are the five long cylindrical bones in the body of the hand that run from the carpal bones of the wrist to the base of each digit of the hand. The ends of the metacarpal bones form the knuckles. In the preferred embodiment the hand support 100 allows a user to support the carpal and metacarpal bones during repetitive tasks.

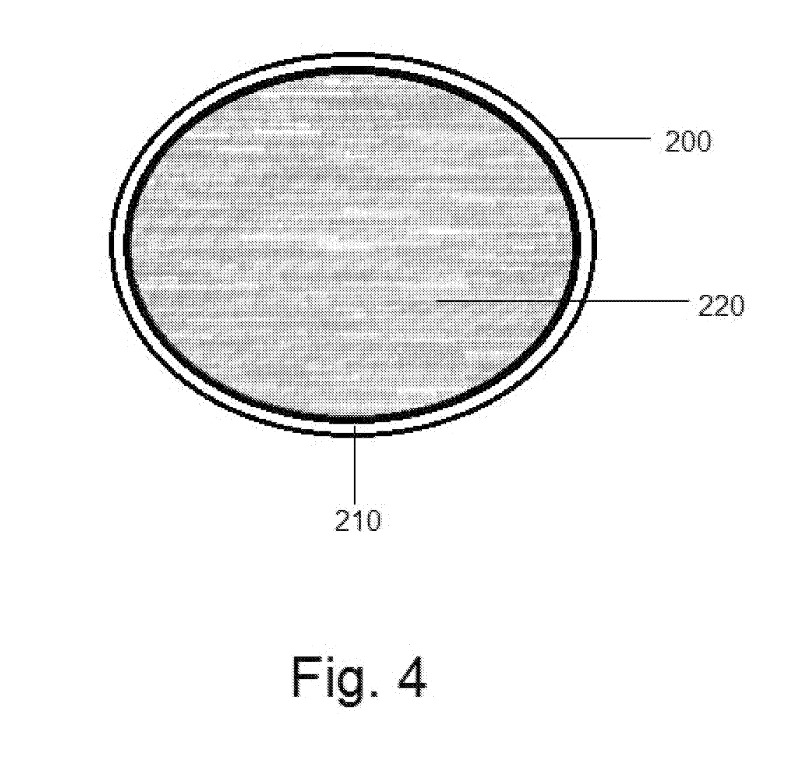

Referring to FIG. 4, a cross section of the hand support 100 is displayed. The shape of the cross section of the hand support 100 may be any shape. In the preferred embodiment the cross sectional shape of the hand support 100 is an oblong shape. The shape of the cross section of the hand support 100 may be circular, oval, oblong, rectangular, diamond, or any other shape. In another embodiment, as displayed in FIG. 4, the hand support 100 may be comprised of an inner material 120 and an outer cover 110. The outer cover 110 is a removable outer encasing for the inner material 120. The outer cover 110 may be any type of material. The outer cover 110 may be form fitting to the inner material 120 or loose. The outer cover 110 may have a simple slit to permit a user to remove and replace the inner material 120. In another embodiment, the outer cover 110 may have a zipper, a plurality of snaps or buttons, or other means to close the outer cover 110 and keep the inner material 120 contained within the outer cover 110. The outer cover 110 may be made from any material. In the preferred embodiment the outer cover 110 is composed of a synthetic fabric to absorb moisture and sweat from the user's hand. Alternatively, the outer cover 110 may be composed of a cotton fabric or synthetic rubber. The inner material 120 may be any type of soft and supportive material. The inner material 120 may be a loose fill contained within the outer cover 110 or may be a preformed body of material. The inner material 120 may be cotton, sponge, synthetic foam, gel, natural fibers, thermoplastic elastomer, or any other soft and compressible material.

The hand support 100 may be any size provided that the tapered, ergonomic shape remains unchanged. The hand support 100 may be a smaller size for female users or a larger size for male users. In the preferred embodiment the hand support 100 is approximately 0.75 inches in thickness at the first end and approximately 0.25 inches in thickness at the second end. In the preferred embodiment the hand support 100 measures between 3.0 inches and 3.5 inches in length from the first end to the second end. In the preferred embodiment the hand support 100 measures approximately 3.5 inches in width at the first end and approximately 2.5 inches in width at the second end.

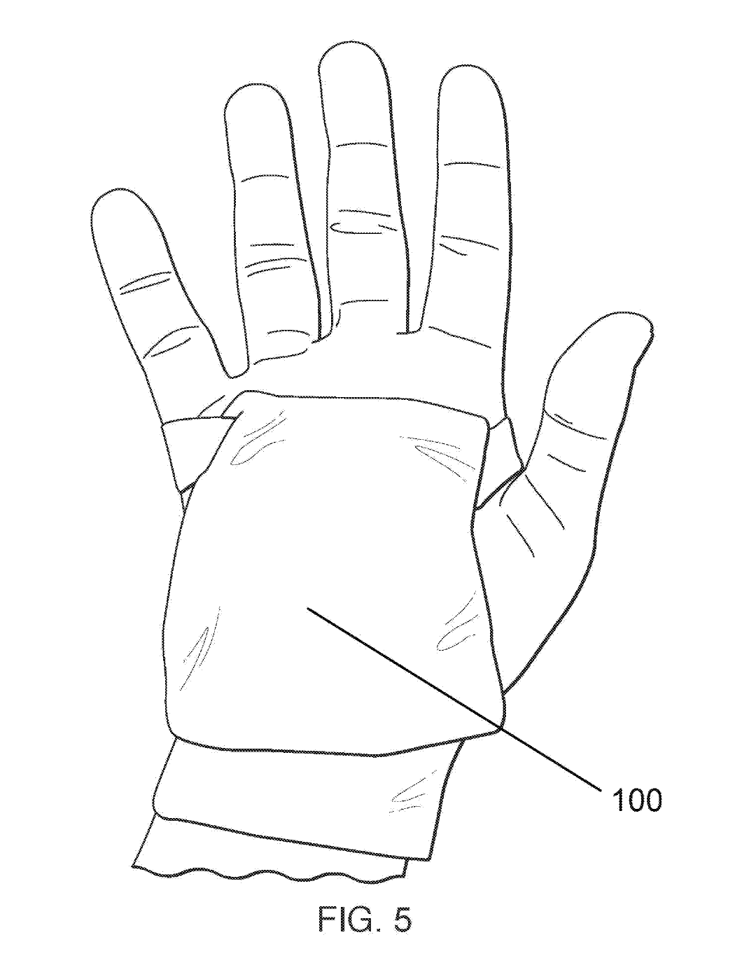

Referring to FIG. 5, an alternative embodiment of the hand support 100 is displayed. In this embodiment, the hand support 100 is not tapered in thickness or in width. The hand support 100 in this embodiment is square or rectangular in shape and has a consistent thickness.

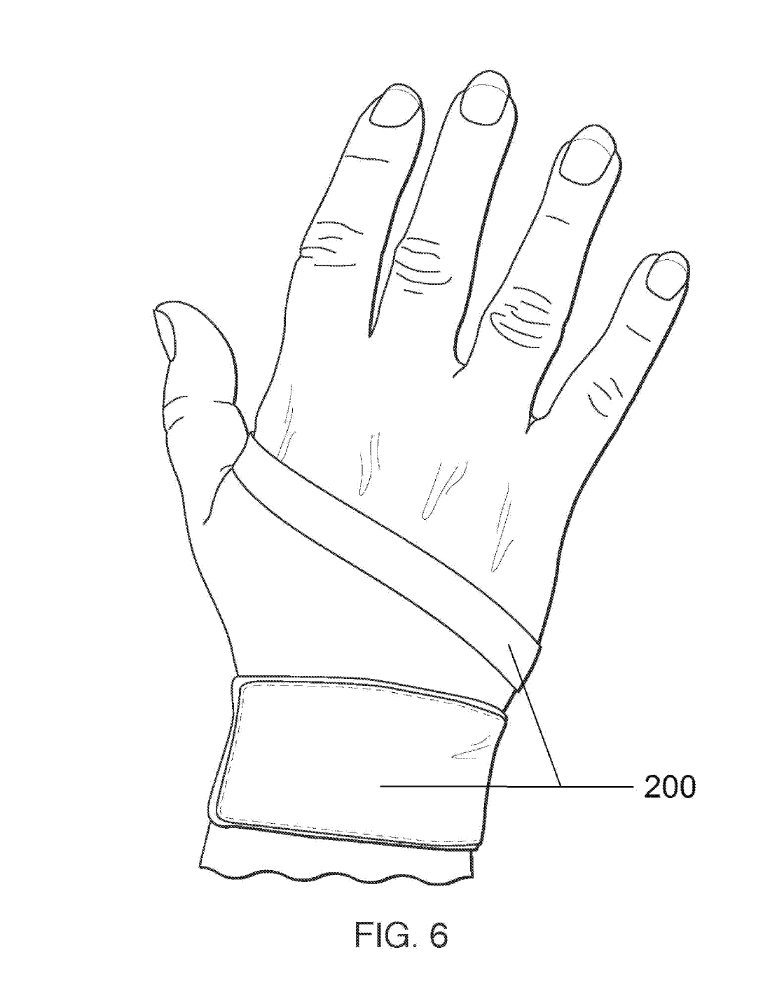

Referring to FIG. 6, an alternative embodiment of the attachment means 200 are displayed. In this embodiment, the attachment means 200 constitutes a looped band wrapping around the wrist of the user and a looped band wrapped around the middle of the hand of the user. The looped band around the wrist is wider and substantially covers the user's wrist.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated regions, integers, steps, operations, elements, and/or components, but do not preclude the presence or addition of one or more other regions, integers, steps, operations, elements, components, and/or groups thereof. Further, any reference to claim elements in the singular, for example, using the articles “a,” “an” or “the” is not to be construed as limiting the element to the singular. As used herein, the terms “and” and “or” shall be interchangeable and include any and all combinations of one or more of the associated listed items.

The claims provided herewith are nonlimiting and for illustrative purposes only. Variations in the claims may exist without departing from the scope of the invention.

1. A wearable device for supporting the wrist of a user during a repetitive task comprising
A hand support
Wherein said hand support comprises a compressible material
Wherein said hand support has a first end and a second end
Wherein said first end of said hand support is thicker and wider than said second end of said hand support
Wherein said hand support is substantially tapered from said first end to said second end
Wherein said first end of said hand support is substantially positioned under the wrist of a user when said hand support is worn by a user
Wherein said second end of said hand support is substantially positioned under the heads of the metacarpal bones when said hand support is worn by the user.
An attachment means
Wherein said attachment means removably secures said hand support to the palm of a user's hand.

2. The wearable device as in claim 1 wherein said attachment means comprises one or more looped bands.

3. The wearable device as in claim 2 wherein said compressible material is selected from a group consisting essentially of cotton, sponge, synthetic foam, gel, natural fibers, or thermoplastic elastomer.

4. The wearable device as in claim 3
Wherein said first end of said hand support is approximately 0.75 inches thick
Wherein said first end of said hand support is approximately 3.5 inches wide
Wherein said second end of said hand support is approximately 0.25 inches thick
Wherein said second end of said hand support is approximately 2.5 inches wide
Wherein the length of said hand support from said first end to said second end is between approximately 3.0 inches and approximately 3.5 inches.

5. The wearable device as in claim 4 further comprising a securing means
Wherein said securing means secures portions of said attachment means together or secures said attachment means to said hand support.

6. The wearable device as in claim 5 wherein said securing means is selected from a group consisting essentially of hook and loop tape, a snap, a button, and a hook and eye.

7. The wearable device as in claim 6
Wherein said attachment means consists of two elastic looped bands
Wherein said two elastic looped bands encircle the back of a user's hand when worn by a user
Wherein said two elastic looped bands overlap at one point on the back of a user's hand.

8. The wearable device as in claim 1 further comprising a securing means
Wherein said securing means secures portions of said attachment means together or secures said attachment means to said hand support.

9. The wearable device as in claim 1
Wherein said compressible material is selected from a group consisting essentially of cotton, sponge, synthetic foam, gel, natural fibers, or thermoplastic elastomer
Wherein said first end of said hand support is approximately 0.75 inches thick
Wherein said first end of said hand support is approximately 3.5 inches wide
Wherein said second end of said hand support is approximately 0.25 inches thick
Wherein said second end of said hand support is approximately 2.5 inches wide
Wherein the length of said hand support from said first end to said second end is between approximately 3.0 inches and approximately 3.5 inches.

10. The wearable device as in claim 1
Wherein said attachment means consists of two elastic looped bands
Wherein said two elastic looped bands encircle the back of a user's hand when worn by a user
Wherein said two elastic looped bands overlap at one point on the back of a user's hand.

11. A wearable device for supporting the wrist of a user during a repetitive task comprising
A hand support
Wherein said hand support comprises
An outer cover
a compressible inner material
Wherein said hand support has a first end and a second end
Wherein said first end of said hand support is thicker and wider than said second end of said hand support
Wherein said hand support is substantially tapered from said first end to said second end
Wherein said first end of said hand support is substantially positioned under the wrist of a user when said hand support is worn by a user
Wherein said second end of said hand support is substantially positioned under the heads of the metacarpal bones when said hand support is worn by the user.
An attachment means
Wherein said attachment means removably secures said hand support to the palm of a user's hand.

12. The wearable device as in claim 11 wherein said compressible inner material is selected from a group consisting essentially of cotton, sponge, synthetic foam, gel, natural fibers, or thermoplastic elastomer.

13. The wearable device as in claim 12 wherein said outer cover is composed of a material selected from a group consisting essentially of a cotton fabric, synthetic fabric, or synthetic rubber.

14. The wearable device as in claim 13 wherein said attachment means comprises one or more looped bands.

15. The wearable device as in claim 14
Wherein said first end of said hand support is approximately 0.75 inches thick
Wherein said first end of said hand support is approximately 3.5 inches wide
Wherein said second end of said hand support is approximately 0.25 inches thick
Wherein said second end of said hand support is approximately 2.5 inches wide
Wherein the length of said hand support from said first end to said second end is between approximately 3.0 inches and approximately 3.5 inches.

16. The wearable device as in claim 15 further comprising a securing means
Wherein said securing means secures portions of said attachment means together or secures said attachment means to said hand support.

17. The wearable device as in claim 16 wherein said securing means is selected from a group consisting essentially of hook and loop tape, a snap, a button, and a hook and eye.

18. The wearable device as in claim 17
Wherein said attachment means consists of two elastic looped bands
Wherein said two elastic looped bands encircle the back of a user's hand when worn by a user
Wherein said two elastic looped bands overlap at one point on the back of a user's hand.

19. The wearable device as in claim 11
Wherein said outer cover is composed of a material selected from a group consisting essentially of a cotton fabric, synthetic fabric, or synthetic rubber.
Wherein said first end of said hand support is approximately 0.75 inches thick
Wherein said first end of said hand support is approximately 3.5 inches wide
Wherein said second end of said hand support is approximately 0.25 inches thick
Wherein said second end of said hand support is approximately 2.5 inches wide
Wherein the length of said hand support from said first end to said second end is between approximately 3.0 inches and approximately 3.5 inches.

20. The wearable device as in claim 11
Wherein said attachment means consists of two elastic looped bands
Wherein said two elastic looped bands encircle the back of a user's hand when worn by a user
Wherein said two elastic looped bands overlap at one point on the back of a user's hand.

21. (canceled)

22. (canceled)
